(19) United States
(12) Patent Application Publication
(10) Pub. No.: US 2016/0175029 A1
(43) Pub. Date: Jun. 23, 2016

(54) TISSUE SEALING AND CUTTING INSTRUMENT WITH LOCKING FEATURES

(71) Applicant: Ethicon Endo-Surgery, Inc., Cincinnati, OH (US)

(72) Inventors: David A. Witt, Maineville, OH (US); Cory G. Kimball, Hamilton, OH (US); David K. Norvell, Monroe, OH (US)

(21) Appl. No.: 14/579,299

(22) Filed: Dec. 22, 2014

(51) Int. Cl. A61B 18/14

(52) U.S. Cl. CPC A61B 18/1445; A61B 2018/1455; A61B 2018/00273

(57) ABSTRACT

A surgical instrument includes a first arm, wherein the first arm comprises a first jaw, and wherein the first jaw includes a first electrically conductive member. The surgical instrument further includes a second arm, wherein the second arm comprises a second jaw, wherein the second jaw includes a second electrically conductive member, wherein the first arm is pivotable relative to the second arm between an open configuration and a closed configuration to capture tissue, and wherein the electrically conductive members are configured to cooperate to deliver energy to the tissue. The surgical instrument further includes a cutting beam, an actuator actuatable proximally to translate the cutting beam distally to sever the tissue, a support structure, and a flexible member extending at least partially around the support structure, and configured to couple the actuator to the cutting beam.

### INTRODUCTION

The present disclosure relates generally to a sealing and cutting forceps and various mechanisms associated therewith.

A variety of surgical instruments include one or more elements that transmit energy, for example radio frequency (RF) energy, to tissue (e.g., to coagulate or seal the tissue). Some such instruments comprise a pair of jaws that open and close on tissue, with conductive tissue contact surfaces that are operable to weld tissue closed between the jaws. In open surgical settings, some such instruments may be in the form of forceps having a scissor grip.

In addition to having RF energy transmission elements, some surgical instruments also include a translating tissue cutting element. Some versions of electrosurgical instruments that are operable to sever tissue may be selectively used in at least two modes. One such mode may include both severing tissue and coagulating tissue. Another such mode may include just coagulating tissue without also severing the tissue. Yet another mode may include the use of jaws to grasp and manipulate tissue without also coagulating and/or severing the tissue.

When an electrosurgical instrument includes grasping jaws and tissue severing capabilities it may be desirable to avoid accidental cutting by the knife. Hence, the instrument may include a feature that prevents the knife from firing until the jaws are sufficiently closed upon the tissue. It may also be desirable to prevent the jaws from being opened until the knife has been retracted. One or both of these features can prevent the knife from being extended while the jaws are open.

Forceps type instruments may in some instances provide a feature that allows the jaws of the forceps to be locked on tissue, so that the operator can remove his or her hands from the instrument. In such an instrument, it may also be desirable to provide a circuit that is activated only when the forceps are closed and sufficient pressure is applied to the tissue between the jaws of the device.

### SUMMARY

In one embodiment, a surgical instrument includes a first arm, wherein the first arm comprises a first jaw, and wherein the first jaw includes a first electrically conductive member. The surgical instrument further includes a second arm, wherein the second arm comprises a second jaw, wherein the second jaw includes a second electrically conductive member, wherein the first arm is pivotable relative to the second arm between an open configuration and a closed configuration to capture tissue between the first jaw and the second jaw, and wherein the first electrically conductive member and the second electrically conductive member are configured to cooperate to deliver energy to the captured tissue. The surgical instrument further includes a cutting beam operable to translate distally relative to the first jaw and the second jaw to sever the tissue captured between the first jaw and the second jaw. The surgical instrument further includes an actuator actuatable to translate the cutting beam distally, a support structure, and a flexible member extending at least partially around the support structure, wherein the flexible member is configured to couple the actuator to the cutting beam, and wherein the actuator is actuatable proximally to translate the cutting beam distally.

In one embodiment, a surgical instrument includes a first arm, wherein the first arm comprises a first jaw, and wherein the first jaw includes a first electrically conductive member. The surgical instrument further includes a second arm, wherein the second arm comprises a second jaw, wherein the second jaw includes a second electrically conductive member, wherein the first arm is pivotable relative to the second arm between an open configuration and a closed configuration to capture tissue between the first jaw and the second jaw, and wherein the first electrically conductive member and the second electrically conductive member are configured to cooperate to deliver energy to the captured tissue. The surgical instrument further includes a cutting beam operable to translate distally relative to the first jaw and the second jaw to sever the tissue captured between the first jaw and the second jaw. The surgical instrument further includes a support structure and a flexible member. The flexible member includes a first end portion and a second end portion coupled to the cutting beam, wherein the cutting beam is pulled distally by the second end portion relative to the support structure in response to a tensioning force applied to the first end portion.

In one embodiment, a surgical instrument includes a first arm, wherein the first arm comprises a first jaw, and wherein the first jaw includes a first electrically conductive member. The surgical instrument further includes a second arm, wherein the second arm comprises a second jaw, wherein the second jaw includes a second electrically conductive member, wherein the first arm is pivotable relative to the second arm between an open configuration and a closed configuration to capture tissue between the first jaw and the second jaw, and wherein the first electrically conductive member and the second electrically conductive member are configured to cooperate to deliver energy to the captured tissue. The surgical instrument further includes a cutting beam and a drive portion operable motivate the cutting beam to translate distally relative to the first jaw and the second jaw to sever the tissue captured between the first jaw and the second jaw. The surgical instrument further includes an actuator actuatable to translate the cutting beam distally, a housing defining a curved path between the actuator and the drive portion, and a flexible member slidably movable through the curved path defined by the housing, wherein the flexible member is configured to couple the actuator to the drive portion, wherein the flexible member is constricted by the housing to permit the flexible member to transmit at least one reciprocating actuation motion through the curved path.

### BRIEF DESCRIPTION OF THE DRAWINGS

The features and advantages provided in this disclosure, and the manner of attaining them, will become more apparent and the disclosure itself will be better understood by reference to the following description of instances of the disclosure taken in conjunction with the accompanying drawings, wherein:

FIG. 1A illustrates a perspective view of one embodiment of a surgical instrument in a closed configuration according to one embodiment;

FIG. 1B illustrates a perspective view of the surgical instrument of FIG. 1A in an open configuration according to one embodiment;

FIG. 2 is a partial cross-sectional view of a proximal portion of the surgical instrument of FIG. 1A according to one embodiment;

FIG. 3 is a rear view of an upper handle ring of the surgical instrument of FIG. 1A illustrating a closure lock arm according to one embodiment;

FIG. 4 is a partial side view of the surgical instrument of FIG. 1A with various parts removed to uncover an actuation mechanism in a default position according to one embodiment;

FIG. 5 is a partial side view of the surgical instrument of FIG. 1A with various parts removed to uncover an actuation mechanism in an actuated position according to one embodiment;

FIG. 6 illustrates a side view of a surgical instrument in accordance with one embodiment; and

FIG. 7 is a partial side view of a surgical instrument with various parts removed to uncover an actuation mechanism in an actuated position according to one embodiment.

Corresponding reference characters indicate corresponding parts throughout the several views. The exemplifications set out herein illustrate various embodiments of the disclosure, in one form, and such exemplifications are not to be construed as limiting the scope of the disclosure in any manner.

### DETAILED DESCRIPTION

The Applicant of the present application also owns the U.S. patent applications identified below which were filed on even date herewith and which are each herein incorporated by reference in their respective entireties:

U.S. patent application Ser. No. ______, entitled RF TISSUE SEALER, SHEAR GRIP, TRIGGER LOCK MECHANISM AND ENERGY ACTIVATION (Attorney Docket No. END7453USNP/140080); and

U.S. patent application Ser. No. ______, entitled RF TISSUE SEALER, SHEAR GRIP, TRIGGER LOCK MECHANISM AND ENERGY ACTIVATION (Attorney Docket No. END7456USNP/140085).

In the following detailed description, reference is made to the accompanying drawings, which form a part hereof. In the drawings, similar symbols and reference characters typically identify similar components throughout the several views, unless context dictates otherwise. The illustrative embodiments described in the detailed description, drawings, and claims are not meant to be limiting. Other embodiments may be utilized, and other changes may be made, without departing from the scope of the subject matter presented here.

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Before explaining the various embodiments of the present disclosure, it should be noted that the various embodiments disclosed herein are not limited in their application or use to the details of construction and arrangement of parts illustrated in the accompanying drawings and description. Rather, the disclosed embodiments may be positioned or incorporated in other embodiments, variations and modifications thereof, and may be practiced or carried out in various ways. Accordingly, embodiments of the surgical devices disclosed herein are illustrative in nature and are not meant to limit the scope or application thereof. Furthermore, unless otherwise indicated, the terms and expressions employed herein have been chosen for the purpose of describing the embodiments for the convenience of the reader and are not to limit the scope thereof. In addition, it should be understood that any one or more of the disclosed embodiments, expressions of embodiments, and/or examples thereof, can be combined with any one or more of the other disclosed embodiments, expressions of embodiments, and/or examples thereof, without limitation.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument.

Also, in the following description, it is to be understood that terms such as front, back, inside, outside, top, bottom, upper, lower and the like are words of convenience and are not to be construed as limiting terms. Terminology used herein is not meant to be limiting insofar as devices described herein, or portions thereof, may be attached or utilized in other orientations. The various embodiments will be described in more detail with reference to the drawings.

An electrosurgical instrument may include a set of jaws, with at least one of the jaws being pivotable relative to the other jaw to selectively compress tissue between the jaws. Once the tissue is compressed, electrically conductive members in the jaws may be activated with bipolar RF energy to seal the tissue. In some instances, a cutting feature is operable to sever tissue that is closed between the jaws. For instance, the cutting feature may be actuated after the RF energy has sealed the tissue. Various references that are cited herein relate to electrosurgical instruments where the jaws are part of an end effector at the distal end of an elongate shaft, such that the end effector and the shaft may be inserted through a port (e.g., a trocar) to reach a site within a patient during a minimally invasive endoscopic surgical procedure. A handpiece may be positioned at the proximal end of the shaft for manipulating the end effector. Such a handpiece may have a pistol grip configuration or some other configuration.

In some instances, it may be desirable to provide an electrosurgical instrument that does not have an elongate shaft or handpiece similar to those described in the various references cited herein. In particular, it may be desirable to provide an electrosurgical instrument that is configured similar to a forceps device, with a scissor grip. Such instruments may be used in a variety of medical procedures. Various examples of electrosurgical shears/forceps devices are disclosed in U.S. Publication No. 2014/0214019, entitled ELECTROSURGICAL HAND SHEARS, published Jul. 31, 2014, the entire disclosure of which is incorporated by reference herein. Various other examples of electrosurgical forceps instruments will be described in greater detail below; while other examples will be apparent to those of ordinary skill in the art in view of the teachings herein.

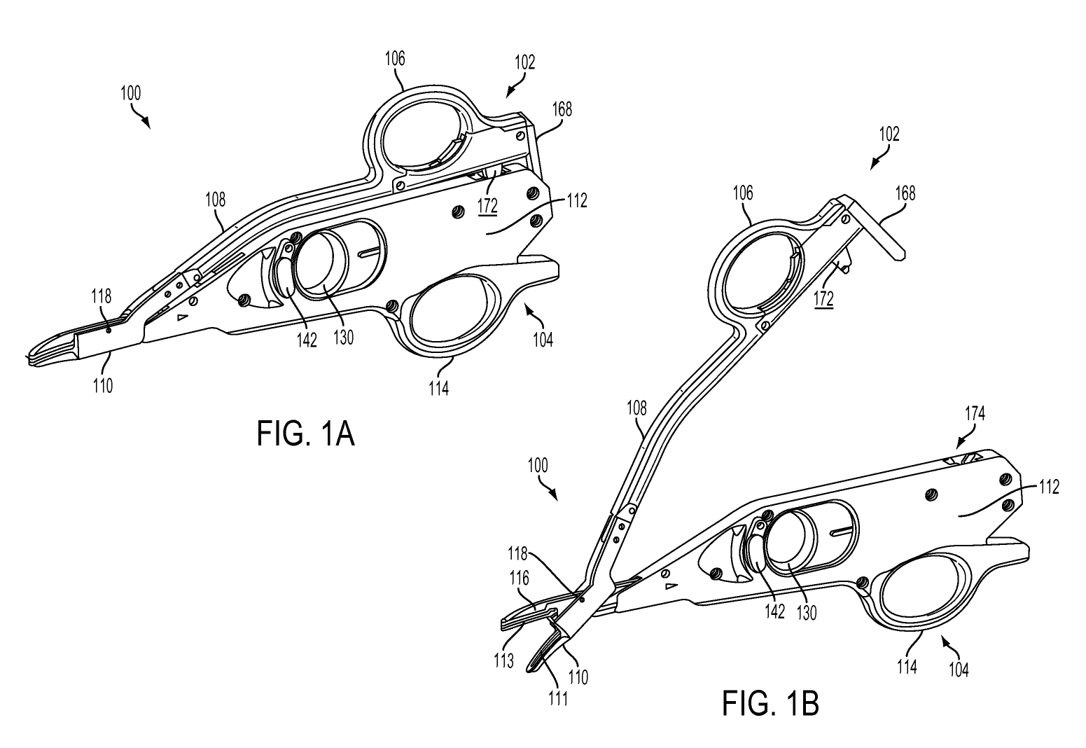

FIG. 1A illustrates a perspective view of one embodiment of a surgical instrument 100 (also called “a cutting forceps” or “an RF cutting forceps”) in a closed configuration. The cutting forceps 100 comprises an upper arm 102 and a lower arm 104 pivotally connected at a pivot joint 118 near the distal end of the device. The upper arm 102 and lower arm 104 are shaped such that the cutting forceps 100 can be operated by either a left-handed or right-handed person. The cutting forceps 100 can also be operated as illustrated or upside down from how it is illustrated. As such, the terms upper and lower and left and right are used for convenience only, and not as a limitation.

The upper arm 102 comprises a first or upper handle ring 106 near the proximal end of the upper arm 102, a bend arm 108 between the proximal and distal ends, and a first or lower jaw 110 at the distal end. The upper handle ring 106 is shaped such that a human finger can be inserted therein. In some embodiments, the upper handle ring 106 comprises a closure lock arm 168 and release arm 172, described in further detail below. The bend arm 108 connects the upper handle ring 106 to the lower jaw 110. The upper handle ring 106, bend arm 108, and lower jaw 110 are connected in a fixed orientation, such that as the upper handle ring 106 is moved, all parts of the upper arm 102 move together.

In certain instances, the lower jaw 110 comprises at least one electrically conductive member 111. Likewise, the upper jaw 116 may include at least one electrically conductive member 113. The electrically conductive members 111, 113 are be configured to transmit energy through tissue positioned, or at least partially positioned, between, or in the vicinity of the electrically conductive members 111, 113 to treat and/or seal the tissue. Energy delivered by electrically conductive members 111, 113 may comprise, for example, radiofrequency (RF) energy, sub-therapeutic RF energy, therapeutic RF energy, ultrasonic energy, and/or other suitable forms of energy.

In certain instances, an energy button 142 can be actuated to transmit energy between the electrically conductive members 111, 113. In certain instances, when the energy button 142 is depressed, a circuit is completed allowing the energy transmission. The circuit may be coupled to a power source. In some embodiments, the power source is a generator. In certain instances, the generator is external to the surgical instrument 100 which is separably coupled to the generator. In other instances, the generator is integrated with the surgical instrument 100. In certain instances, the power source may be suitable for therapeutic tissue treatment, tissue cauterization/sealing, as well as sub-therapeutic treatment and measurement.

The lower arm 104 comprises a lower arm body 112 and a second or upper jaw 116. Integrated with the proximal end of the lower arm body 112 is a second or lower handle ring 114. The lower handle ring 114 is shaped such that a human finger can be inserted therein. The distal end of the lower arm body 112 is connected to the upper jaw 116. The lower arm body 112, the lower handle ring 114, and the upper jaw 116 are connected in a fixed orientation such that all parts of the lower arm 104 move together. The lower arm body 112 further comprises an actuator 130 for controlling the operation of a cutting member 120, described in further detail below. In some embodiments, the lower arm body 112 also comprises the energy button 142 for activating the RF energy, also described in further detail below.

FIG. 1B illustrates a perspective view of the cutting forceps 100 shown in FIG. 1A in an open configuration. As explained above, the upper arm 102 is pivotally connected at a pivot joint 118 to the lower arm 104 near the distal end of the cutting forceps 100. As the upper arm 102 is raised, the proximal end of the upper arm 102 pivots away from the lower arm 104. At the same time, the lower jaw 110 pivots away from the upper jaw 116, thus opening the jaws 110, 116. The motion of the upper arm 102 relative to the lower arm 104 can also be described as a scissor motion. The upper arm 102 can be lowered to return the cutting forceps 100 to the closed configuration as illustrated in FIG. 1A.

In some embodiments, the lower arm 104 includes a closure lock arm slot 402 for receiving the closure lock arm 168. The closure lock arm 168 may extend or protrude from the upper arm 102 and can be guided by the upper arm 102 into the closure lock arm slot 402 as the upper arm 102 is moved toward the lower arm 104. Embodiments including the closure lock arm 168 and the closure lock arm slot 402 are described in further detail below. In some embodiments, the lower arm 104 includes a release arm slot 174 for receiving a release arm 172. The release arm 172 may extend or protrude from the upper arm 102 and can be guided by the upper arm 102 into the release arm slot 174 as the upper arm 102 is moved toward the lower arm 104. Embodiments including the release arm 172 and the release arm slot 174 are described in further detail below.

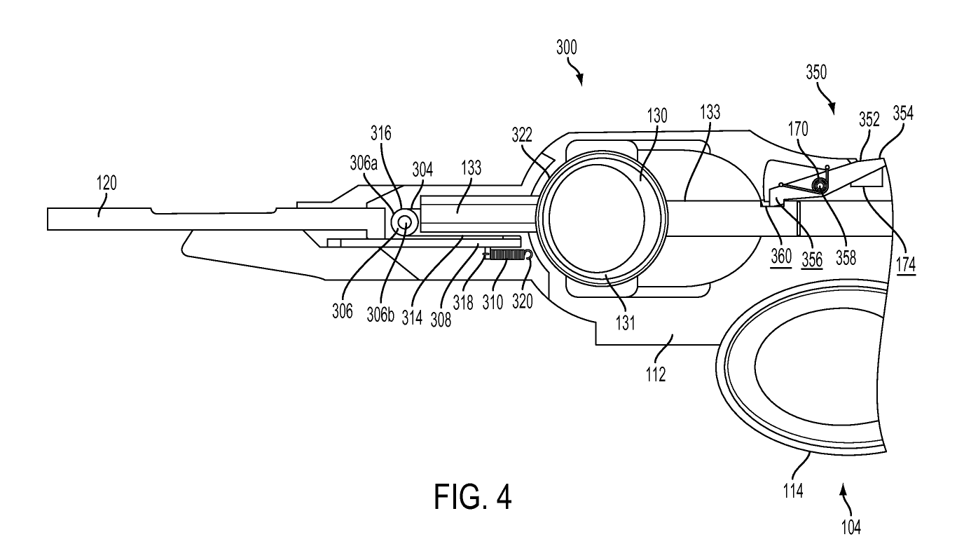

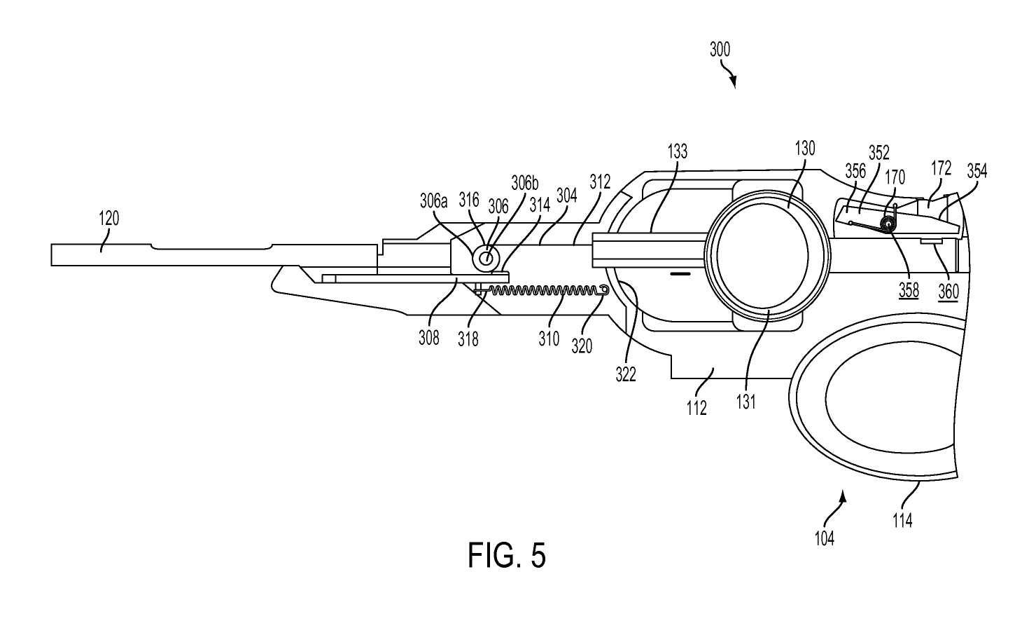

FIGS. 4 and 5 depict a self-resetting actuation mechanism 300 employed with the cutting forceps 100 to actuate a cutting member 120. FIG. 4 illustrates the actuation mechanism 300 in an unactuated default or starting configuration. FIG. 5 illustrates the actuation mechanism 300 in an actuated configuration. In the embodiment illustrated in FIGS. 4 and 5, the actuation mechanism 300 includes an actuator 130, a flexible member 304, a support member 306, a drive portion 308, and a biasing member 310. In the embodiment illustrated in FIGS. 4 and 5, the cutting member 120 extends from the drive portion 308. The actuator 130 can be actuated from the default configuration, as illustrated in FIG. 4, to the actuated configuration, as illustrated in FIG. 5, to drive the cutting member 120 distally to cut tissue captured between the jaws 110, 116.

In the embodiment illustrated in FIGS. 4 and 5, the flexible member 304 includes a first end portion 312 and a second end portion 314. The first end portion 312 is coupled to the actuator 130 and the second end portion 314 is coupled to the drive portion 308. An intermediate portion 316 extending between the first end portion 312 and the second end portion 314 is supported by the support member 306. The intermediate portion 316 may extend around or about the support member 306 to transmit at least one actuation motion between the first end portion 312 and the second end portion 314. The first end portion 312 can be pulled or retracted proximally by the actuator 130 to cause the second end portion 314 to be advanced or translated distally thereby causing the drive portion 308 and, in turn, the cutting member 120 to be advanced or translated distally.

In certain instances, the support member 306 comprises a bearing surface 306a. The flexible member 304 is slidably movable relative to the bearing surface 306a. In certain instances, the support member 306 is fixedly attached to the lower arm body 112. In certain instances, the support member 306 is rotatable about a central axis transecting the lower arm body 112. In at least one example, the support member 306 rotates about an axle 306b fixedly attached to the lower arm body 112. In such instances, the rotation of the support member 306 may facilitate the sliding motion of the flexible member 304.

The support member 306 can be configured to reverse, or at least change, a direction of motion of the flexible member 304. For example, as described above, the second end portion 314 is slidably movable distally in response to the proximal motion of the first end portion 312. Such an arrangement can be advantageous in converting proximal actuation motions applied to the actuator 130 to distal drive motions of the cutting member 120. This motion conversion allows an operator of the cutting forceps 100 to deploy the cutting member 120 by retracting the actuator 130, which can be easily achieved using the same hand operating the ring handles 106 and 114.

In certain instances, at least one of the intermediate portion 316 of the flexible member 304 and the bearing surface 306a is coated with a material that enhances the sliding motion of the flexible member 304 relative to the bearing surface 306a. For example, at least one of the intermediate portion 316 of the flexible member 304 and the bearing surface 306a is coated with polytetrafluoroetheylene (PTFE), also known as Teflon.

In at least one example, the support member 306 comprises a cylindrical shape which has a circumferential rim that contains the bearing surface 306a. The intermediate portion 316 can be extended at least partially around the circumferential rim of the support member 306. Other shapes and configurations of the support member 306 are contemplated by the present disclosure.

In certain instances, the actuator 130 can be comprised of a pull ring 131 and an actuation plate 133. In certain instances, as illustrated in FIG. 4, the pull ring 131 is integrated with the actuation plate 133. In the embodiment illustrated in FIG. 4, the actuation plate 133 is coupled to the first end portion 312 of the flexible member 304. The actuation plate 133 can be mounted within the lower arm body 112 such that it is able to slide along the proximal-distal axis of the cutting forceps 100. In the default configuration of the actuation mechanism 300, the pull ring 131 rests against the internal wall 322. When the biasing member 310 is in a relaxed or minimally biased orientation, the pull ring 131 is in a distal or default position; that is the pull ring 131 is further away from the operator of the device and the cutting member 120 is retracted. As the pull ring 131 is retracted proximally, the flexible member 304 motivates the drive portion 308 to translate the cutting member 120 distally to cut tissue captured between the upper jaw 116 and lower jaw 110.

The relative position of the ring handle 106, the ring handle 114, and the pull ring 131 permits a surgical operator to operate the cutting forceps 100 with one hand. The operator can actuate the ring handles 106 and 114 to capture tissue and actuate the pull ring 131 to advance the cutting member to cut the captured tissue using a single hand, which frees the other hand to operate other surgical instruments, for example.

In the embodiment illustrated in FIGS. 4 and 5, the biasing member 310 is employed to reset the actuation mechanism 300 to a default configuration. The biasing member 310 includes a first end 318 coupled to the drive portion 308 and a second end 320 coupled to the lower arm body 112. As described above, retraction of the actuator 130 proximally causes the drive portion 308 to translate distally. Translation of the drive portion 308 distally causes the biasing member 310 to be expanded or stretched, as illustrated in FIG. 5. Upon release of the actuator 130, the biasing member 310 returns the actuation mechanism 300 to the default configuration by returning the drive portion 308 and, in turn, the actuator 130 to their default positions, as illustrated in FIG. 4. As the drive portion 308 is retracted to the default position by the biasing member 310, the drive portion 308 returns the flexible member 304 to a default position by retracting the second end portion 314 to its default position, which causes the first end portion 312 and, in turn, the actuator 130, to be advanced distally to their default positions.

In certain instances, the flexible member 304 is maintained under tension while the actuation mechanism 300 is in the default configuration. In one example, the biasing member 310 is maintained in a slightly biased configuration while the actuation mechanism 300 is in the default configuration, which causes the biasing member 310 to apply a threshold biasing force against the drive portion 308, the flexible member 304 and, in turn, the actuator 130. The tension created by the threshold biasing force can remove or reduce any slack in the flexible member 304. In addition, the threshold biasing force can abut the actuator 130 against the internal wall 322 of the lower arm body 112 in the default configuration of the actuation mechanism 300, as illustrated in FIG. 4. In certain instances, a proximal end of the drive portion 308 may rest against an internal wall of the lower arm body 112 in the default configuration of the actuation mechanism 300.

The above-described arrangement can provide a safety advantage by preventing, or at least resisting, incidental advancement of the cutting member 120 in response to unintended contact with the actuator 130. To advance the cutting member 120, the actuator 130 needs to be retracted with a force greater than the threshold biasing force. The biasing force of the biasing member 310 can also provide tactile feedback to an operator of the actuator 130.

In certain instances, the biasing member 310 can be a tension coil spring. In such instances, the tension coil spring can be slightly stretched to set the threshold biasing force to a desired pre-load. In certain instances, the biasing member 310 can be a compression coil spring. In such instances, the tension coil spring can be slightly compressed to set the threshold biasing force to a desired pre-load. In at least one example, the biasing member 310 can be a torsion spring. In certain instances, the biasing member 310 can be comprised, or at least partially comprised, of an elastic material.

The actuation mechanism 300 can be particularly useful in transmitting actuation motions in tight locations. Unlike other actuation mechanisms, the actuation mechanism 300 occupies very limited space and can be effectively operated to transmit actuation motions around corners, for example. This can be particularly desirable in the surgical world where surgical instruments are deigned to work in tight spaces and, as such, need to be small in size. Furthermore, the actuation mechanism 300 is relatively light in weight compared to other actuation mechanisms. This is also desirable in the surgical world. Surgical instruments can be held by surgical operators for prolonged time intervals. A surgical instrument equipped with the actuation mechanism 300 can be lighter and easier to hold than a similar surgical instrument equipped with another actuation mechanism.

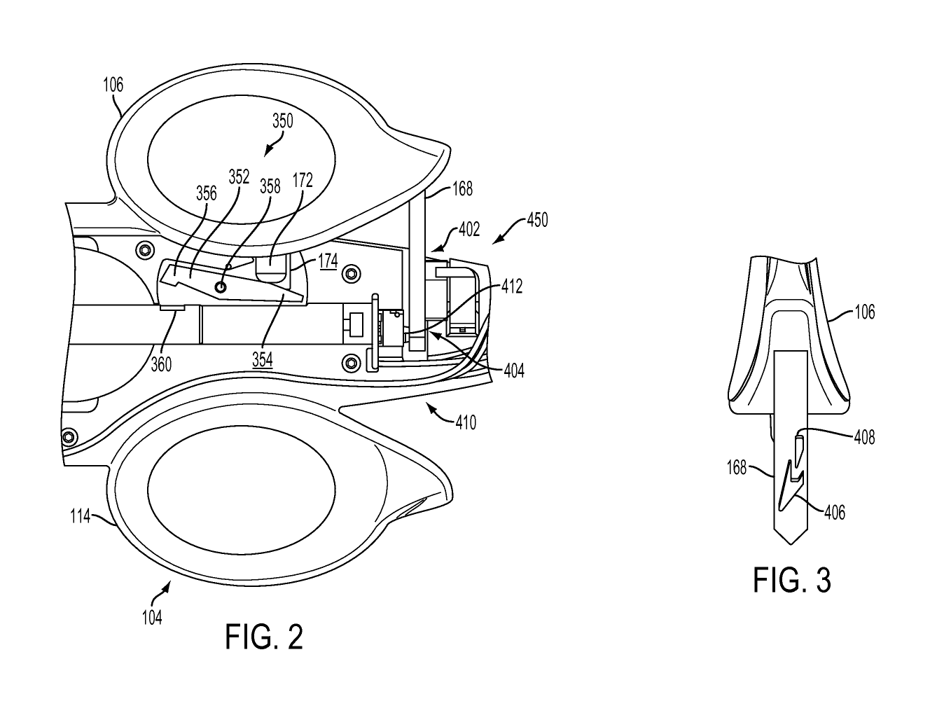

FIG. 2 illustrates the components of an embodiment of an actuator lockout mechanism 350 that may be included in some embodiments of the cutting forceps 100. The actuator lockout mechanism 350 comprises a release arm 172, a lock spring 170, a release arm slot 174, a toggle member 352, and an actuator slot 360. FIG. 4 illustrates the actuator lockout mechanism 350 in a locked configuration. The toggle member 352 is engaged with the actuator 130 in the locked configuration to resist or prevent actuation of the actuation mechanism 300. FIG. 5 illustrates the actuator lockout mechanism 350 in an unlocked configuration. The actuator 130 is released from the toggle member 352 in the unlocked configuration to permit actuation of the actuation mechanism 300. In certain instances, as described in greater detail below, the actuator 130 is released from the toggle member 352 while the cutting forceps 100 is in the closed configuration.

In the embodiment illustrated in FIGS. 4 and 5, the toggle member 352 is movable about an axis defined by a pin 358 fixedly attached to the lower arm body 112. A first end portion 354 and a second end portion 356 of the toggle member 352 can be situated on opposite sides of the pin 358. The toggle member 352 can be rotated about the pin 358 between a lock position and a release position. In the lock position, the second end portion 356 of the toggle member 352 may hookingly engage the actuator slot 360, as illustrated in FIG. 4, to prevent or resist translation of the actuator 130. The lock spring 170, which can be a torsion spring, may bias the toggle member 352 in the lock position.

The toggle member 352 is transitioned to the release position by applying a force to the first end portion 354 that overcomes the biasing force of the lock spring 170. The force applied to the first end portion 354 may cause the toggle member 352 to be rotated about the pin 358 thereby releasing the second end portion 356 from the actuator slot 360, as illustrated in FIG. 5. Freed from the toggle member 352, the actuator 130 can be retracted to advance the cutting member 120, as described above.

In certain instances, transitioning the cutting forceps 100 to the closed configuration may cause the actuator lockout mechanism 350 to be transitioned to the unlocked configuration. In certain instances, the closure of the jaws 110, 116 and the release of the lockout mechanism 350 can be achieved concurrently. Such an arrangement can be advantageous in safeguarding against incidental deployment of the cutting member 120 while the cutting forceps 100 is in the open configuration. As illustrated in FIG. 4, the first end portion 354 of the toggle member 352 may be situated, or at least partially situated, in the release arm slot 174. As the upper arm 102 is transitioned toward the lower arm 104 to close the jaws 110, 116, the release arm 172 follows a predetermined trajectory leading into the release arm slot 174. The predetermined trajectory brings the release arm 172 into contact with the first end portion 354. The release arm 172 may motivate the toggle member 352 to release the actuator 130 by moving or depressing the first end portion 354.

Released from the toggle member 352, the actuator 130 can be retracted to advance the cutting member 120 to cut tissue captured by the jaws 110, 116 in the closed configuration of the cutting forceps 100. Coupling the release of the actuator lockout mechanism 350 to the closure mechanism of the cutting forceps 100 reduces the steps required to operate the cutting forceps 100, which is particularly advantageous with the single-handed operation of the cutting forceps 100; a separate release switch for the lockout mechanism 350 would require additional effort or even a second hand to release the lockout mechanism 350.

FIG. 2 illustrates the components of an embodiment of a combination closure lock and energy activation mechanism 450 that may be included in some embodiments of the cutting forceps 100. The mechanism 450 comprises a closure lock arm 168, a slot 402, a lock arm catch member or pin 404, a first cam groove 406, a second cam groove 408, a compression circuit 410, and a compression circuit button 412. It should be noted that although the combination closure lock and energy activation mechanism 450 as described herein uses similar elements for the closure lock and the energy activation, it is understood that not all embodiments require all the elements described. In some embodiments, only a closure lock mechanism is desired. Such embodiments may comprise some but not necessarily all of elements of the combination mechanism. In other embodiments, only an energy activation mechanism is desired. Likewise, such embodiments may comprise some but not all the elements of the combination mechanism. Yet other embodiments may include separate components for a closure lock mechanism and an energy activation mechanism.

As described above, the release arm 172 and the closure lock arm 168 may both extend or protrude from the upper arm 102. In certain instances, the release arm 172 and the closure lock arm 168 are simultaneously guided into their respective slots by the upper arm 102 as the upper arm 102 is moved to transition the cutting forceps 100 to the closed configuration. In at least one example, the trajectories of the release arm 172 and the closure lock arm 168 as the upper arm 102 is moved toward the lower arm 104 are in parallel with each other. In at least one example, the release arm 172 defines a first axis intersecting the upper arm 102 and the closure lock arm 168 defines a second axis intersecting the upper arm 102, wherein the first axis is in parallel with the second axis.

The positions of the release arm 172 and the closure lock arm 168 relative to each other, relative to the upper arm 102, relative to the lower arm 104, and relative to their respective slots can be adjusted to coordinate the entries of the release arm 172 and the closure lock arm 168 into their respective slots. In at least one example, the closure lock arm 168 is configured to enter a first closed position at the same time the actuator 130 is released from the toggle member 352 by the release arm 172. In at least one example, the closure lock arm 168 is configured to enter the first closed position slightly before the actuator 130 is released from the toggle member 352 by the release arm 172. In such examples, the actuator 130 can be retracted to deploy the cutting member 120 while the closure lock arm 168 is in the first closed position.

In certain instances, the closure lock arm 168 is further driven by the upper arm 102 to enter a second closed position following the first closed position. The second closed position can be a final closed position. In at least one instance, the closure lock arm 168 is configured to enter the second closed position at the same time the actuator 130 is released from the toggle member 352 by the release arm 172. In at least one instance, the closure lock arm 168 is configured to enter the second closed position slightly before the actuator 130 is released from the toggle member 352 by the release arm 172. In such instances, the actuator 130 can be retracted to deploy the cutting member 120 while the closure lock arm 168 is in the second closed position but not the first closed position.

The cutting forceps 100 may be transitioned between the open configuration, the first closed position, and the second closed position. The cutting forceps 100 can be employed as a surgical clamp while in the first closed position. An operator can actuate the cutting forceps 100 to capture and hold tissue such as, for example, a blood vessel by transitioning the cutting forceps 100 to the first closed position thereby closing the jaws 110, 116 around the tissue. The cutting forceps 100 can be locked in the first closed position to maintain hold of the captured tissue. To release the captured tissue, the cutting forceps 100 can be unlocked from the first closed position to permit the jaws 110, 116 to transition to the open configuration.

In certain instances, the cutting forceps 100 can be locked in the first closed position by locking the closure lock arm 168 in the first closed position. The lock arm catch member or pin 404, which extends from the lower arm 104 into the slot 402, can be configured to lock the closure lock arm 168 in the first closed position. In at least one instance, the closure lock arm 168 includes a first cam groove 406 which has a heart-shaped configuration, as illustrated in FIG. 3. In such instances, to lock the closure lock arm 168 in the first closed position, the pin 404 is navigated through the heart-shaped configuration of the first cam groove to a locked position by the advancement of the closure lock arm 168 through the slot 402.

To unlock the closure lock arm 168, an operator may further push the upper arm 102 toward the lower arm 104 slightly depressing the closure lock arm 168 deeper into the slot 402. In result, the pin 404 is navigated out of the locked position of the first cam groove 406. The upper arm 102 may then be moved away from the lower arm 104 thereby returning the cutting forceps 100 to the open configuration to release the captured tissue. Alternatively, to further transition the closure lock arm 168 to the second closed position, additional pressure may be applied to move the upper arm 102 even further toward the lower arm 104. In result, the pin 404 is navigated into the second cam groove 408.

The mechanism 450 can be configured to allow energy transmission between the electrically conductive members 111, 113 while the jaws 110, 116 are in the closed configuration but not while the jaws 110, 116 are in the open configuration. In certain instances, the mechanism 450 can be configured to allow energy transmission between the electrically conductive members 111, 113 while the closure lock arm 168 is in the second closed position but not while the closure lock arm 168 is in the first closed position. In such instances, the allowance of energy transmission may coincide with the transition of the closure lock arm 168 to the second closed position.

The closure lock arm 168 may be configured to depress the compression circuit button 412 as the closure lock arm 168 the second closed position. The compression circuit button 412 can be operably coupled to the compression circuit 410. In certain instances, the compression circuit 410 can be coupled to a power source (not shown) and the electrically conductive members 111, 113. In certain instances, depression of the compression circuit button 412 by the closure lock arm 168 closes the compression circuit 410 thereby allowing energy transmission between the electrically conductive members 111, 113 while the closure lock arm 168 is in the second closed position.

In certain instances, the compression circuit 410 also includes the energy button 142. In such instances, energy transmission between the electrically conductive members 111, 113 requires transitioning the closure lock arm 168 to the second closed position and further actuating the energy button 142 to close the compression circuit 410.

In certain instances, the cutting forceps 100 can be employed to perform a “cold” cut, wherein tissue captured by the cutting forceps 100 is cut without or prior to the passing of energy through the captured tissue. In such instances, the cutting forceps 100 is actuated to the first closed position to capture the tissue between the jaws 110, 116. In the first closed position, the first cam groove 406 of the closure lock arm 168 can be locked with the pin 404 to maintain the cutting forceps 100 in the first closed position. Furthermore, in such instances, the actuator 130 is released from the toggle member 352 such that it can be actuated to deploy the cutting member 120 to cut the captured tissue. No energy can be passed between the electrically conductive members 111, 113 because the compression circuit button 412 remains undepressed.

In certain instances, the cutting forceps 100 can be employed to perform a “hot” cut, wherein tissue captured by the cutting forceps 100 is sealed and cut. In such instances, the cutting forceps 100 is actuated to the second closed position free the actuator 130 and depress the compression circuit button 412. An operator may then depress the energy button 142 to seal and/or treat the captured tissue and retract the actuator 130 thereby deploying the cutting member 120 to cut the captured tissue.

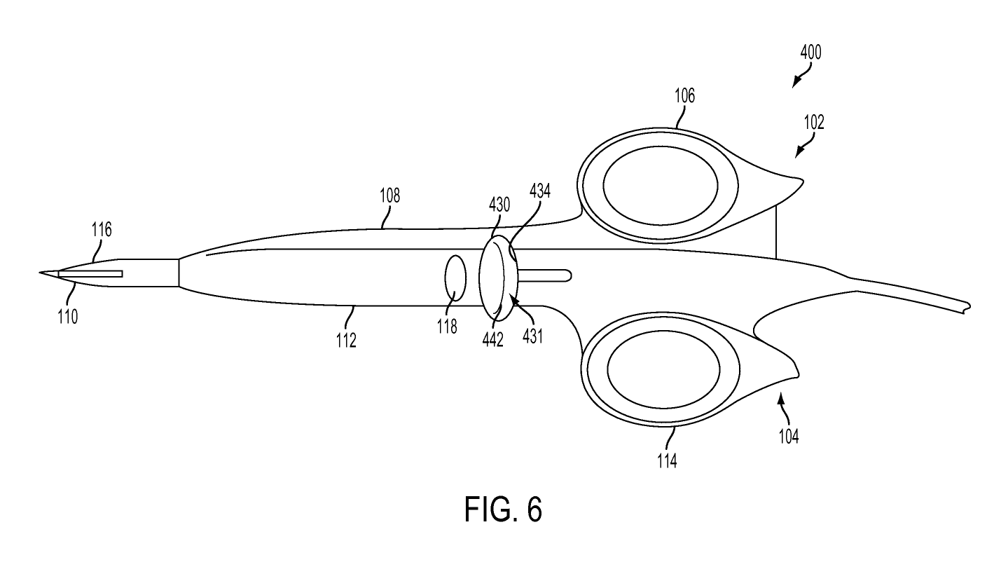

FIG. 6 illustrates one embodiment of a surgical instrument 400. The surgical instrument 400 is similar in many respects to the cutting forceps 100. For example, like the cutting forceps 100, the surgical instrument 400 comprises an upper arm 102 and a lower arm 104 pivotally connected at a pivot joint 118. In addition, like the cutting forceps 100, the surgical instrument 400 comprises a first or upper handle ring 106, a second or lower handle ring 114, an upper arm body 108, a lower arm body 112, an upper jaw 116, and a lower jaw 110.

Further to the above, like the cutting forceps 100, the surgical instrument 400 comprises the self-resetting actuation mechanism 300. In the embodiment illustrated in FIG. 6, an energy button 442 is integrated with an actuator 430. Otherwise, the actuator 430 is similar in many respects to the actuator 130. For example, like the actuator 130, the actuator 430 is coupled to the flexible member 304 and is retracted proximally to advance the cutting member 120 distally. The energy button 442 is similar in many respects to the energy button 142. Integration of the energy button 442 with the actuator 430 simplifies the operation of the surgical instrument 400. An operator of the surgical instrument 400 does not need to toggle or switch between the actuator 430 and the energy button 442. The operator of the surgical instrument 400 may depress the energy button 442 to transmit energy through tissue captured by the jaws 110, 116, and then retract the actuator 430 proximally to advance the cutting member 120 to cut the captured tissue without significant effort and without the need to change or reorient the operator's grip on the surgical instrument 400.

In at least one instance, the actuator 430 includes a socket 431. The energy button 442 can be embedded in the socket 431, as illustrated in FIG. 6. An operator, while gripping the handle 106, 114, may insert an index finger into the socket 431 to depress the energy button 442 deeper into the socket 431 from a default position. Depressing the energy button 442 may cause energy to be transmitted through tissue captured by the jaws 110, 116. In one embodiment, the energy transmission can be stopped automatically after a predetermined time interval regardless of whether the operator continues to depress the energy button 442. In another embodiment, the operator may slightly relieve the pressure on the energy button 442 to stop the energy transmission. In any event, the index finger can then press against a proximal wall 434 of the socket 431 to retract the actuator 430 proximally to advance the cutting member 120 to cut the captured tissue.

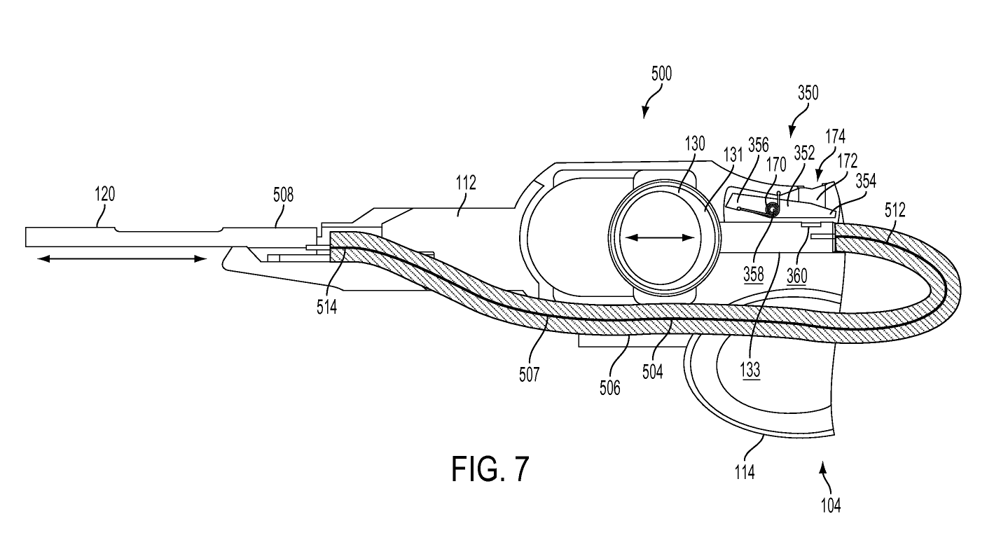

FIG. 7. 7 illustrates one embodiment of an actuation mechanism 500 employed with the cutting forceps 100 to actuate the cutting member 120. In certain instances, the actuation mechanism 500 can be employed with other surgical instruments such as, for example, the surgical instrument 400. The actuator mechanism 500 is similar in many respects to the actuation mechanism 300. For example, like the actuation mechanism 300, the actuator mechanism 500 comprises the actuator 130. In the embodiment depicted in FIG. 7, the actuator 130 is coupled to a flexible member 504.

Like the flexible member 304, the flexible member 504 includes a first end portion 512 and a second end portion 514. In the embodiment depicted in FIG. 7, the first end portion 512 is coupled to the actuator 130 and the second end portion 514 is coupled to a drive portion 508. The flexible member 504 extends through a support member 506. In certain instances, the support member 506 can be flexible. In at least one example, the support member 506 is less flexible than the flexible member 504 extending therethrough.

In certain instances, the support member 506 may tightly accommodate or constrict the flexible member 504 to increase the column strength of the flexible member 504 to permit the flexible member 504 to transmit a compressive force between the first end portion 512 and the second end portion 514. In the embodiment depicted in FIG. 7, the actuator 130 can be retracted, once it is released from the toggle member 352, to translate the cutting member 120 distally. In certain instances, the actuator 130 can be translated between a default position and an actuated position, as illustrated in FIG. 7, to cause the flexible member 504 to likewise translate through the support member 506. Translation of the flexible member 504 may cause the drive portion 508 and, in turn, the cutting member 120 to likewise translate between an undeployed position and a deployed position, as illustrated in FIG. 7.

In certain instances, the support member 506 comprises a tubular, or at least substantially tubular, shape. The flexible member 504 may comprise a cable that extends through the hollow space defined by the support member 506. An inner wall 507 of the support member 506 may closely surround the flexible member 504 to prevent, or at least resist, collapse of the flexible member 504 under the compressive forces applied thereto by the actuator 130 as the actuator 130 is retracted to deploy the cutting member 120. In at least one instance, a lubricant may be employed to facilitate the translation of the flexible member 504 relative to the support member 506. In at least one instances, an adjustment member (not shown) can be employed to adjust the length of the flexible member 504.

The various mechanisms of the present disclosure are described in connection with a cutting forceps for illustrative purposes only. The various mechanisms described herein such as, for example, the mechanism 300, the mechanism 350, and the mechanism 450 can be utilized with various other surgical instruments in open and minimally invasive surgical procedures. For example, the actuation mechanism 300 can be employed with a laparoscopic or an endoscopic surgical instrument. In one embodiment, the actuation mechanism 300 can be employed with a surgical stapler (not shown) to deploy a plurality of staples. For example, the drive portion 308 can be coupled to a sled which can be motivated by the drive portion 308 to deploy the plurality of staples in response to actuation motions applied to the actuator 130.

In some cases, various embodiments may be implemented as an article of manufacture. The article of manufacture may include a computer readable storage medium arranged to store logic, instructions and/or data for performing various operations of one or more embodiments. In various embodiments, for example, the article of manufacture may comprise a magnetic disk, optical disk, flash memory or firmware containing computer program instructions suitable for execution by a general purpose processor or application specific processor. The embodiments, however, are not limited in this context.

The functions of the various functional elements, logical blocks, modules, and circuits elements described in connection with the embodiments disclosed herein may be implemented in the general context of computer executable instructions, such as software, control modules, logic, and/or logic modules executed by the processing unit. Generally, software, control modules, logic, and/or logic modules comprise any software element arranged to perform particular operations. Software, control modules, logic, and/or logic modules can comprise routines, programs, objects, components, data structures and the like that perform particular tasks or implement particular abstract data types. An implementation of the software, control modules, logic, and/or logic modules and techniques may be stored on and/or transmitted across some form of computer-readable media. In this regard, computer-readable media can be any available medium or media useable to store information and accessible by a computing device. Some embodiments also may be practiced in distributed computing environments where operations are performed by one or more remote processing devices that are linked through a communications network. In a distributed computing environment, software, control modules, logic, and/or logic modules may be located in both local and remote computer storage media including memory storage devices.

Additionally, it is to be appreciated that the embodiments described herein illustrate example implementations, and that the functional elements, logical blocks, modules, and circuits elements may be implemented in various other ways which are consistent with the described embodiments. Furthermore, the operations performed by such functional elements, logical blocks, modules, and circuits elements may be combined and/or separated for a given implementation and may be performed by a greater number or fewer number of components or modules. As will be apparent to those of skill in the art upon reading the present disclosure, each of the individual embodiments described and illustrated herein has discrete components and features which may be readily separated from or combined with the features of any of the other several aspects without departing from the scope of the present disclosure. Any recited method can be carried out in the order of events recited or in any other order which is logically possible.

Unless specifically stated otherwise, it may be appreciated that terms such as “processing,” “computing,” “calculating,” “determining,” or the like, refer to the action and/or processes of a computer or computing system, or similar electronic computing device, such as a general purpose processor, a DSP, ASIC, FPGA or other programmable logic device, discrete gate or transistor logic, discrete hardware components, or any combination thereof designed to perform the functions described herein that manipulates and/or transforms data represented as physical quantities (e.g., electronic) within registers and/or memories into other data similarly represented as physical quantities within the memories, registers or other such information storage, transmission or display devices.

It is worthy to note that some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, also may mean that two or more elements are not in direct contact with each other, but yet still co-operate or interact with each other. With respect to software elements, for example, the term “coupled” may refer to interfaces, message interfaces, and application program interface (API), exchanging messages, and so forth.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

Any patent, publication, or other disclosure material, in whole or in part, that is the to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is the to be incorporated by reference herein, but which conflicts with existing definitions, statements, or other disclosure material set forth herein will only be incorporated to the extent that no conflict arises between that incorporated material and the existing disclosure material.

While this invention has been described as having example designs, the present invention may be further modified within the spirit and scope of the disclosure. This application is therefore intended to cover any variations, uses, or adaptations of the invention using its general principles. Further, this application is intended to cover such departures from the present disclosure as come within known or customary practice in the art to which this invention pertains.

The entire disclosures of:

U.S. patent application Ser. No. 12/576,789, entitled SURGICAL INSTRUMENT FOR TRANSMITTING ENERGY TO TISSUE COMPRISING NON-CONDUCTIVE GRASPING PORTIONS, filed Oct. 9, 2009, now U.S. Pat. No. 8,747,404;
U.S. patent application Ser. No. 14/075,839, entitled ELECTROSURGICAL DEVICES, filed Nov. 8, 2013;
U.S. patent application Ser. No. 14/075,863, entitled ELECTROSURGICAL DEVICES, filed Nov. 8, 2013; and
U.S. patent application Ser. No. 14/229,033, entitled DISTAL SEALING END EFFECTOR WITH SPACERS, filed Mar. 28, 2014, are hereby incorporated by reference herein.

What is claimed is:

1. A surgical instrument, comprising:
a first arm, wherein the first arm comprises a first jaw, and wherein the first jaw includes a first electrically conductive member;
a second arm, wherein the second arm comprises a second jaw, wherein the second jaw includes a second electrically conductive member, wherein the first arm is pivotable relative to the second arm between an open configuration and a closed configuration to capture tissue between the first jaw and the second jaw, and wherein the first electrically conductive member and the second electrically conductive member are configured to cooperate to deliver energy to the captured tissue;
a cutting beam operable to translate distally relative to the first jaw and the second jaw to sever the tissue captured between the first jaw and the second jaw;
an actuator actuatable to translate the cutting beam distally;
a support structure; and
a flexible member extending at least partially around the support structure, wherein the flexible member is configured to couple the actuator to the cutting beam, and wherein the actuator is actuatable proximally to translate the cutting beam distally.

2. The surgical instrument of claim 1, further comprising a lock configured to selectively restrict translation of the cutting beam.

3. The surgical instrument of claim 2, wherein the cutting beam is free from the lock in the closed configuration.

4. The surgical instrument of claim 1, wherein the support structure comprises a bearing surface, and wherein the flexible member is slidably movable over the bearing surface.

5. The surgical instrument of claim 1, further comprising a biasing member configured to return the cutting beam to a default position.

6. The surgical instrument of claim 5, wherein the cutting beam comprises a proximal portion, and wherein the biasing member is coupled to the proximal portion of the cutting beam.

7. The surgical instrument of claim 1, further comprising a clamp arm lock configured to lock the first arm and the second arm in the closed configuration.

8. The surgical instrument of claim 1, further comprising an energy switch, wherein the closed configuration comprises:
a first closed position, wherein, in the first closed position, electrical energy is not permitted to flow between the first electrically conductive member and the second electrically conductive member in response to activation of the energy switch; and
a second closed position, wherein, in the second closed position, energy is permitted to flow between the first electrically conductive member and the second electrically conductive member in response to activation of the energy switch.

9. The surgical instrument of claim 8, wherein the actuator comprises the energy switch.

10. A surgical instrument, comprising:
a first arm, wherein the first arm comprises a first jaw, and wherein the first jaw includes a first electrically conductive member;
a second arm, wherein the second arm comprises a second jaw, wherein the second jaw includes a second electrically conductive member, wherein the first arm is pivotable relative to the second arm between an open configuration and a closed configuration to capture tissue between the first jaw and the second jaw, and wherein the first electrically conductive member and the second electrically conductive member are configured to cooperate to deliver energy to the captured tissue;
a cutting beam operable to translate distally relative to the first jaw and the second jaw to sever the tissue captured between the first jaw and the second jaw;
a support structure; and
a flexible member, comprising:
a first end portion; and
a second end portion coupled to the cutting beam, wherein the cutting beam is pulled distally by the second end portion relative to the support structure in response to a tensioning force applied to the first end portion.

11. The surgical instrument of claim 10, further comprising a locking feature configured to selectively restrict translation of the cutting beam.

12. The surgical instrument of claim 11, wherein the cutting beam is free from the locking feature in the closed configuration.

13. The surgical instrument of claim 10, further comprising an energy switch, wherein the closed configuration comprises:
a first closed position, wherein, in the first closed position, electrical energy is not permitted to flow between the first electrically conductive member and the second electrically conductive member in response to activation of the energy switch; and
a second closed position, wherein, in the second closed position, energy is permitted to flow between the first electrically conductive member and the second electrically conductive member in response to activation of the energy switch.

14. The surgical instrument of claim 10, wherein the support structure comprises a bearing surface, and wherein the flexible member is slidably movable over the bearing surface.

15. The surgical instrument of claim 10, further comprising a biasing member configured to return the cutting beam to a default position.

16. The surgical instrument of claim 15, wherein the cutting beam comprises a proximal portion, and wherein the biasing member is coupled to the proximal portion of the cutting beam.

17. The surgical instrument of claim 10, further comprising a clamp arm lock configured to lock the first arm and the second arm in the closed configuration.

18. A surgical instrument, comprising:
a first arm, wherein the first arm comprises a first jaw, and wherein the first jaw includes a first electrically conductive member;
a second arm, wherein the second arm comprises a second jaw, wherein the second jaw includes a second electrically conductive member, wherein the first arm is pivotable relative to the second arm between an open configuration and a closed configuration to capture tissue between the first jaw and the second jaw, and wherein the first electrically conductive member and the second electrically conductive member are configured to cooperate to deliver energy to the captured tissue;
a cutting beam;
a drive portion operable motivate the cutting beam to translate distally relative to the first jaw and the second jaw to sever the tissue captured between the first jaw and the second jaw;
an actuator actuatable to translate the cutting beam distally;
a housing defining a curved path between the actuator and the drive portion; and
a flexible member slidably movable through the curved path defined by the housing, wherein the flexible member is configured to couple the actuator to the drive portion, wherein the flexible member is constricted by the housing to permit the flexible member to transmit at least one reciprocating actuation motion through the curved path.

19. The surgical instrument of claim 18, further comprising a locking feature configured to selectively restrict translation of the cutting beam.

20. The surgical instrument of claim 19, wherein the cutting beam is free from the locking feature in the closed configuration.
